(19) United States
(12) Patent Application Publication
(10) Pub. No.: US 2004/0009138 A1
(43) Pub. Date: Jan. 15, 2004

(54) Skin cleanser compositions and methods of use

(76) Inventor: William O. Kling, Dallas, TX (US)

Correspondence Address: JENKENS & GILCHRIST, PC, 1445 ROSS AVENUE, SUITE 3200, DALLAS, TX, 75202, US

(21) Appl. No.: 10/440,395

(22) Filed: May 19, 2003

Related U.S. Application Data
(60) Provisional application No. 60/381,926, filed on May 20, 2002.

(51) Int. Cl. A61K 7/06; A61K 7/11

(52) U.S. Cl. 424/70.12; 424/70.1

(57) ABSTRACT

The present invention relates to a liquid cleansing product for topical application that may be left on the applied area without requiring to be rinsed off with water. When used topically, the cleansing product effectively reduces the level of sebum and microbes on the skin in a relatively short time.

### CROSS REFERENCES TO RELATED APPLICATIONS

This application claims priority to U.S. Provisional Application Serial No. 60/381,926 filed on May 20, 2002, which is fully incorporated by reference herein.

### BACKGROUND OF THE INVENTION

1. Technical Field of the Invention

The present invention relates to skin cleanser compositions that are useful for topical application, for the cleansing of hair, skin, nails and adjacent tissue of humans and animals. The present invention also relates to methods of using skin cleanser compositions on hair, skin, nails and adjacent tissue.

2. Description of the Prior Art

It is well known that cleansing one's hands with soap and hot water is an effective means of cleaning the skin surface as well as reducing microorganisms. However, if the hands are not thoroughly dried, the trace water residue can harbor bacteria and fungi, such as yeast and mold. While this may not pose a serious threat to the population at large, in a hospital setting where the population is more prone to infection, it is desirable to minimize any contamination risks.

In the past few years, research efforts have been directed toward formulating a liquid cleansing product that will effectively clean and sanitize skin, hair and nails without the use of washing with water. Many of these liquid cleansing products incorporate relatively high concentrations or weight percentages of organic alcohols in the compositions and other ingredients such as benzalkonium chloride, benzethonium chloride, hexylresorcinol and tincture of iodine. The alcohols allow the product to dry quickly, but also cause the skin to dehydrate to an unacceptable degree, irritating the skin and causing it to crack and chafe. In addition, they are flammable, are not effective against spore-forming fungi or spore-forming bacteria and may not adequately cleanse the skin of oils, such as sebum.

There is therefore a need to develop leave-on, topical, liquid cleansing compositions that do not require water for effectiveness when applied to skin or hair and are more gentle to skin or hair, and more effective against a broad range of microorganisms.

### SUMMARY OF THE INVENTION

The present invention relates to a liquid cleansing product for topical application that may be left on the applied area without requiring to be rinsed off with water. When used topically, the cleansing product effectively reduces the level of unwanted oils and microbes on the skin in a relatively short time. Further, the cleansing product dries quickly without causing damage and drying to the skin or hair with repeated use. The cleansing product may comprise water soluble moisturizing agents and thereby provide a moisturizing benefit to the skin.

The present invention provides a cleanser composition comprising an effective amount of a chlorine dioxide compound. The cleanser composition may further comprise an agent to further the drying process, and emollients or oils for skin moisturizing. The present invention also relates to methods for reducing the level of oils (sebum) and microorganisms on the skin using the leave-on cleanser compositions described herein.

### DETAILED DESCRIPTION OF ILLUSTRATIVE EMBODIMENTS

The present invention relates to liquid cleansing compositions for use on skin, hair and nails of humans and animals, including animal fur.

The compositions of the present invention comprise an effective amount of a chlorine dioxide compound that is efficacious in removing sebum and organic debris, such as dead skin cells, from the area of application via oxidation. The chlorine dioxide compound present in the cleansing compositions of the present invention also removes microorganisms from the area of application via an oxidation process.

The term “cleanser” as used herein is intended to refer to a composition that is capable of removing oil/sebum and organic debris from the area of application, removing microorganisms from the area of application, and oxidizing the area of application. Similarly the term “cleansing” as used herein is intended to refer to the removal of oil/sebum and organic debris from the area of application of the cleanser, removal of microorganisms from the area of application, and oxidation of the area of application.

In an embodiment of the invention, the cleanser compositions of the present invention are used to clean wounds and regions of the skin that have undergone a trauma. As used herein, the term “wound” is intended to refer to a condition involving trauma to the skin and/or external body surface, and includes, but is not limited to the following conditions: areas of the skin where the skin surface is broken (not intact), external surface burns, pressure ulcers, surgical wounds abrasions and other trauma. In these embodiments, the cleanser composition assists in the reduction or elimination of undesirable microorganisms from the treated area, and further assists in the removal of dead tissue without exacerbating the wound area. The cleansing of the wound area occurs via an oxidation process, which in turn promotes healing.

Certain embodiments of the invention comprise an agent which aids in the drying process. In certain embodiments of the invention, the drying agent is a silicone component. Other embodiments of the invention comprise emollients which moisturize and refresh the skin. Other embodiments of the invention optionally comprise one or more ingredients found in skin cleansers including, but not limited to, benzalkonium chloride, benzethonium chloride, hexylresorcinol, tincture of iodine, isopropyl alcohol and methylbenzethonium chloride or a combination thereof.

The liquid cleansers of the present invention comprise one or more chlorine dioxide compounds to provide antimicrobial functionality, as well as to assist in the removal of sebum from the area of application. Examples of such chlorine dioxide compounds include, but are not limited to, sodium chlorite, sodium chlorate and chlorite ion. The terms “chlorine dioxide generating compound” and “chlorine dioxide compound” are used interchangeably herein. In an embodiment of the invention, the chlorine dioxide compound is an aqueous solution comprising chlorine dioxide. The aqueous solution is prepared by dissolving chlorine dioxide gas in purified water.

The liquid cleanser compositions of the present invention may be in the form of a skin cleanser, exfoliating scrub cleanser, micro-dermabrasion product, hand sanitizer or shampoo for humans and animals.

In an embodiment of the invention, the concentration of chlorine dioxide compound present in the compositions ranges from about 0.005 wt % to about 0.5 wt %. In an alternate embodiment of the invention, the concentration of the chlorine compound ranges from about 0.01 to about 0.4 wt %. In yet another embodiment of the invention, the concentration of the chlorine dioxide compound varies from about 0.03 wt % to about 0.15 wt %.

One or more silicone based materials may be optionally included in the liquid cleanser compositions of the present invention to further aid in the drying process. The silicone based materials, such as cyclomethicone, trimethylsiloxy silicate or a combination thereof, may be included in the formulation at a concentration of from about 5 wt % to about 35 wt %. In certain embodiments of the invention, the drying agent is ethyl alcohol, which is incorporated at levels of less than 10% v/v and is well below the level of alcohol in flammable products.

In addition, humectants may be optionally added to assist in the retention of liquids within the liquid cleanser composition of the present invention, and thickening agents may be added to modify the viscosity of the liquid cleanser composition. In an embodiment of the invention, the humectant included at a concentration of from about 0 wt % to about 5 wt % and the thickener is included at a concentration of from about 0 wt % to about 6.5 wt %. The thickener may be a cellulose-based material, fumed silica, or a combination thereof, such as methyl cellulose added at concentrations of about 0 wt % to about 1.5 wt % used in combination with fumed silica added at concentrations of about 0 wt % to about 5.0 wt %. Other thickeners that may be used in embodiments of the invention include, carbomers which are high molecular weight polymers comprising polyacrylic acid backbones.

Optionally, emollients and aesthetic additives, such as fragrance and/or colorants may also be added to the liquid cleanser formulation. Emollients or moisturizing agents, fragrance and colorants are added as necessary and at concentrations for consumer acceptance. For example, the formulation may include from about 0 wt % to about 1.5 wt % fragrance, dye or a combination thereof. The formulation may also include from about 0 wt % to about 5 wt % of water soluble moisturizing agents such as glycerin or methoxy aminopropyl PEG/PPG-7/13 dimethicone polyol or a combination thereof. The moisturizing agents are added to the cleansing product to aid in the moisturization of the area of application.

An embodiment of the invention provides a method of cleansing an external body surface of a human or animal comprising topical application of a cleanser composition of the present invention.

The following examples are representative of the liquid cleansers which can be prepared in accordance with the present invention. Representative antimicrobial performance data and sebum removal data of certain of the cleansers are also included. The cleanser formulations presented are intended for example purposes only and are not intended to be limiting in scope.

### WORKING EXAMPLES

A. Determination of the Antimicrobial Efficacy of a Cleanser Composition

Purpose:

The purpose of this study is to evaluate the antimicrobial effectiveness of a test product using the following procedure.

Scope:

The antimicrobial effectiveness of a test product utilizing four (4) human subjects per test product at each application time over the course of ten (10) consecutive product applications, with microbial samples taken at baseline and after product application one (1), three (3), seven (7), and ten (10). Serratia marcescens (ATCC #14756) is as the marker organism.

Test Material:

Hand Sanitizer Cleanser

Equipment:

Pipetter 1.0 mL Capacity

Pipetter 0.1 mL Capacity

Bunsen Burner

Clock with Second Hand

Incubator 25°+/−2° C.

Vortex Mixer

Refrigerator, 2°-8° C.

Supplies:

Sterile 5.0 mL Capacity Serological Pipettes

Sterile Dilution Tubes

Sterile Polystyrene Petri Dishes

Sterile Powder-Free Surgical Gloves

Sterile 1.0 mL Capacity Pipette Tips

Sterile 0.1 mL Capacity Pipette Tips

70% Ethanol

Propane Gas Bottles

Test Tube Racks

Test Solutions and Media:

Sampling Solution

Sterile Stripping Fluid (SSF)

Neutralizing and Diluting Fluid

Butterfield's Phosphate Buffer Solution with Product Neutralizers (BBP+)

Sterile Stripping Fluid with Product Neutralizers (SSF+)

Media

Tryptic Soy Agar (TSA)

Tryptic Soy Broth (TSB) for Neutralization Assay and Inoculum Preparation

Test Methods:

Four (4) test subjects used one (1) test product over the course of ten (10) product utilization procedures.

Overtly healthy subjects over the age of eighteen (18), but under the age of seventy (70) were admitted to the study. All subjects' hands were free from clinically evident dermatoses, injuries to the hands and forearms, open wounds, hangnails, and/or any other disorders, which may compromise the subjects and the study. No subjects were admitted into the study if they were known to be using any topical or systemic antimicrobial, steroids, or any other medication known to affect the normal microbial flora of the skin.

Inoculum Preparation

Serratia marcescens (ATCC#14756) was used to challenge the efficacy of the test product. A stock culture of Serratia marcescens was prepared by transferring one colony from an agar plate or slant aseptically to 10 mL of Sterile Trypticase Soy Broth (TSB), which then was incubated at 25°+/−2° C. for 24+/−2 hours. A 1-liter flask containing 500 mL of TSB was inoculated with 0.5 mL of the 24-hour broth, transferred and incubated for 20+/−2 hours at 25°+/−2° C. Prior to any withdrawal of culture, whether for hand contamination or for numbers assay, the suspension was stirred or shaken. The suspension was assayed for number of organisms at the beginning and at the end of the study use period. The suspension was not used more than six hours.

Neutralization

Prior to initiation of the study, Serratia marcescens was used to confirm the adequacy of the antimicrobial product neutralizer in accordance with Standard Practice for Evaluation Inactivators of Antimicrobial Agents Used in Disinfectants, Sanitizers, Antiseptic, or Preserved Products (ASTM E 1054-91).

Test Period

Each subject was utilized for two (2) or three (3) hours on a single day for the test period.

A practice wash was performed using bland soap to remove dirt and oil from the hands and to familiarize the subjects with the test product wash procedure. The temperature of the water used for this, and all subsequent wash cycles was controlled at 40°+/−2° C.

On the designated test day and test phase, a 5.0 mL aliquot of the suspension containing approximately 1.0×10⁸ cfu/mL of Serratia marcescens (ATCC#14756) was transferred into each subject's cupped hands. The inoculum wasthen be distributed evenly over both hands, and not reaching above the wrist, via gentle continuous massage for forty-five (45) seconds. After a timed two (2) minute air-dry, the subjects utilized the test product according to the directions below.

1. Five (5) mLs of test product was dispensed into subject's cupped hands.

2. Subjects rubbed hands together and lathered for thirty (30) seconds.

3. Subjects rinsed their hands for thirty (30) seconds.

4. Subjects lightly dried their hands with a disposable paper towel.

Each subject will completed the above-described inoculation/product application cycle a total of ten (10) consecutive times, with a minimum of five (5), and a maximum of fifteen (15) minutes between each cycle. The hands were sampled for residual Serratia marcescens after inoculation/product application cycles one (1), three (3), seven (7), and ten (10). All samples will be taken using the Glove Juice Sampling Procedure detailed below.

Glove Juice Sampling Procedure

Following the prescribed product application procedure, powder-free, loose fitting sterile latex gloves were donned. At the designated sampling time, seventy-five (75) mL of Sterile Stripping Fluid without product neutralizers were instilled into the glove. The wrist was secured, and the hand was massaged through the glove in a uniform manner for sixty (60) seconds. A 5.0 mL aliquot of the glove juice (dilution 10°) was removed and serial diluted in Sterile Stripping Fluid with product neutralizers and Butterfield's Phosphate Buffer solution.

Plating

Duplicate serial plates were prepared from appropriate dilutions using Tryptic Soy Agar. The plates were incubated at 25°+/−2° C. for approximately forty-eight (48) hours. Serratia marcescens produce red colonies, and only those colonies were counted.

Method of Analysis:

Data Collection

The number of viable microorganisms recovered was estimated by using the formula, 75×Dilution Factor×Mean Plate Count for the two (2) plates. The estimated log₁₀ number of viable microorganisms recovered from each hand was designated the “R-Value.” It is the adjusted average log₁₀ colony count measurement for each subject at each sampling time. Each R-value was determined using the following formula:

R=log₁₀[75×Cᵢ×10^{-D}×2]

where:

75=the amount of stripping solution instilled into each glove

Cᵢ=the arithmetic average colony count of the two (2) plate counts for each subjects at a particular dilution level

D=the dilution factor

2=the neutralization dilution.

The difference of the log of the inoculum level and the R-value, is the log reduction or V-Value, was determined using the following formula:

V=Log I−R-Value

where:

I=the inoculum level of microorganisms

R=R-value as calculated above

Findings and Conclusions:

A two-log reduction for hand sanitizers utilizing the glove juice test showed significant antibacterial activity. Three of the panelists started the study simultaneously. The fourth panelist started the study three hours later. This is noted because the initial inoculum counts were higher for panelist number four than for the first three test subjects. The log reduction for panelist number four was also higher than the first three panelists.

The hand sanitizer showed a 2.4 average log reduction of Serralia marcescens for the first three panelists for the four test washes. Panelist number four showed an average 4.2 log reduction for the four test washes.

The glove juice test results show that the hand sanitizer was effective in reducing the Serratia marcescens and shows significant antibacterial activity.

B. Sebum Reduction Study

Purpose:

The purpose of this study is to evaluate the effectiveness of a test product in reducing sebum on human hair and skin.

Scope:

The test product was evaluated using three adult subjects of varying age, sex and skin condition.

Test Material:

Test Product: Waterless Skin Cleanser and Shampoo

Equipment:

Sebumeter SM 810

Supplies:

Paper toweling

Test Method & Findings:

Four (3) test subjects used one (1) test product, with three areas of hair and three areas of skin examined and treated per subject.

Test On Intact Skin

The Sebumeter measures micrograms of sebum (a microgram is one-millionth of a gram) per square centimeter. Sebum readings were taken on three facial skin areas for each individual, with an initial average for all sites on all subjects of 209 micrograms per site.

A cotton pad saturated with product was used to apply the test solution to the selected skin areas which had been measured. The skin was allowed to air dry over approximately a four minute period. Measurements were taken again in the same locations by the same machine operator. The sebum readings after application of the cleanser averaged 47, for a 78% average sebum reduction on skin.

Test On Hair

Sebum readings were taken on the same subjects in the same way on three areas of the hair and scalp. An initial average for all sites on all subjects was 176. Test solution was sprayed on the hair, rubbed in briskly for approximately one minute by the respondent, then dried with paper towel by the respondent, followed by air drying as much as possible over approximately another two minutes. The sebum readings after application of the cleanser averaged 30, for an 83% reduction in sebum level on the hair.

Conclusions

Based upon a 78% reduction in sebum level on skin and an 83% reduction on hair over approximately a four minute period, we conclude the chlorine dioxide cleanser solution is very effective in reducing sebum levels without conventional washing with soap and/or water.

C. Laboratory Studies of Cleanser

I Objective:

To demonstrate that the test product demonstrates the preservative and/or antimicrobial properties of the label claim.

II References:

21 C.F.R. §333. Topical antimicrobial drug products for over-the counter human use. Microconsult, Inc. Test Method MC-14. Antiseptic Testing for OTC Drug Products.

III Test Organisms:

Cultures of the following microorganisms are maintained as stock cultures from which working inoculum are prepared. The viable microorganisms used in this test must not be more than five passages removed from the original stock culture. For purposes of the test, one passage is defined as the transfer of organisms from an established culture to fresh medium. All transfers are counted.

A. Escherichia coli (ATCC No. 8739)

B. Staphylococcus aureus (ATCC No. 6538)

IV Materials:

A. Test tubes with closures

B. Pipettes, 10.0 ml and 1.0 ml serological

C. 0.85% Phosphate buffered saline or peptone water, pH 7.0-7.2

D. Petri dishes, culture loops, and other microbiological apparatus

V Media:

A. Tryptic Soy Agar with lecithin and Tween 80

VI Procedure:

A. Preparation of Test Samples:

1. Accurately pipette 9.9 ml of product into each of six appropriately labeled or coded test tubes.

2. Store test samples at ambient temperature.

B. Preparation of Inoculum:

1. Inoculate the surface of a suitable volume of solid agar medium from a recently grown stock culture of each of the specified microorganisms. Incubate the bacterial cultures at 30-35° C. for 18-24 hours

2. To harvest the bacterial cultures, place a loop full of the test microorganisms from the plate into the tube containing saline and vortex. Adjust the count with sterile saline or additional microorganisms so that the concentration of the inoculum level is between 10⁻⁷ and 10⁻⁸ microorganisms per ml of product.

3. Determine the number of viable microorganisms in each milliliter of the inoculum suspensions by serial dilution in sterile phosphate buffered saline:

4. Plate dilutions of 10⁻⁶, 10⁻⁷ and 10⁻⁸ for all organisms.

5. Overlay with approximately 20 ml of 45° C. Tryptic Soy Agar with lecithin and Tween 80.

6. Incubate for 48 hours at 30-35° C. for both test organisms.

7. Count test organisms.

8. Calculate the number of organisms as colony forming units per ml (cfu/ml) of inoculum as follows:

$$\frac{\text{cfu/ml} ( 0.1 ml )}{9.9 ml} = \text{cfu/ml of product}$$

C. Inoculation and Plating of Samples:

1. Aseptically transfer 0.1 ml of each test suspension into the appropriately labeled 9.9 ml sample of test material. Each test organism is inoculated as a pure culture into a single 9.9 ml sample of test material.

2. Thoroughly mix or stir all samples by vortex.

3. Let stand for 15 seconds and 5.0 minutes.

4. Remove aliquots at 15 seconds and 5.0 minutes and transfer to 9.9 ml sterile saline.

5. Perform serial dilutions from 10⁻² to 10⁻⁶

6. Transfer 1.0 ml of each dilution into a 100×15 mm petri plate in duplicate.

7. Overlay with approximately 20 ml of 45° C. Tryptic Soy Agar with lecithin and Tween 80.

8. Gently swirl plates and allow to solidify.

9. Incubate plates for 48 hours at 35° C. and 48 hours at 25° C.

D. Sample Evaluation:

1. Read plates and record results on appropriate data sheet.

2. Using the calculated inoculum concentration of each test microorganism, calculate the log reduction of each microorganism for each kill rate.

VII Data:

A. Kill rate Results

1. 15-second Results

| | E. coli ATCC 8739 | | S. aureus ATCC 6538 | |
|---|---|---|---|---|
| Inoculum level | 2.50 × 10⁷ | | 2.05 × 10⁷ | |
| Direct | 0 | 0 | 0 | 0 |
| 10⁻² | 0 | 0 | 0 | 0 |
| 10⁻⁴ | 0 | 0 | 0 | 0 |
| 10⁻⁵ | NA | NA | NA | NA |

| | | |
|---|---|---|
| Average Count | 0 | 0 |
| Log Reduction | 7 | 7 |

2. 5-Minute Results

| | E. coli ATCC 8739 | | S. aureus ATCC 6538 | |
|---|---|---|---|---|
| Inoculum level | 2.50 × 10⁷ | | 2.05 × 10⁷ | |
| Direct | 0 | | 0 | |
| 10⁻² | 0 | | 0 | |
| 10⁻⁴ | 0 | | 0 | |
| 10⁻⁵ | NA | NA | NA | NA |

| | | |
|---|---|---|
| Average Count | 0 | 0 |
| Log Reduction | 7 | 7 |

What is claimed is:

1. A liquid cleanser composition comprising from about 0.005 wt % to about 0.5 wt % of a chlorine dioxide compound, wherein said cleanser is suitable for application on skin, hair and nails of humans and animals, and animal fur.

2. The composition of claim 1 further comprising a drying agent.

3. The composition of claim 2 wherein said drying agent is a silicone based material.

4. The composition of claim 1 wherein said drying agent is ethyl alcohol.

5. The composition of claim 1 further comprising a humectant.

6. The composition of claim 1 further comprising a thickening agent.

7. The composition of claim 1 further comprising a fragrance.

8. The composition of claim 7 wherein said fragrance is added at a concentration of from about 0 wt % to about 1.5 wt %.

9. The composition of claim 1 further comprising a colorant.

10. The composition of claim 1 further comprising one or more moisturizing agents.

11. The composition of claim 10 wherein said one or more moisturizing agents are added at a concentration of from about 0 wt % to about 5 wt %.

12. The composition of claim 1 further comprising one or more of benzalkonium chloride, benzethonium chloride, hexylresorcinol, tincture of iodine, isopropyl alcohol and methylbenzethonium chloride or a combination thereof.

13. The composition of claim 1 wherein said composition is skin cleanser, exfoliating scrub cleanser, micro-dermabrasion product, hand sanitizer or shampoo for humans and animals.

14. A method of cleansing an external body surface of a human or animal comprising topical application of a composition, wherein said composition comprises from about 0.005 wt % to about 0.5 wt % of a chlorine dioxide compound.

15. The method of claim 14 wherein said composition further comprises a drying agent.

16. The method of claim 15 wherein said drying agent is a silicone based material.

17. The method of claim 15 wherein said drying agent is ethyl alcohol.

18. The method of claim 14 wherein said composition further comprises a humectant.

19. The method of claim 14 wherein said composition further comprises a thickening agent.

20. The method of claim 14 wherein said composition further comprises a fragrance.

21. The method of claim 14 wherein said composition further comprises a colorant.

22. The method of claim 14 wherein said composition further comprises one or more moisturizing agents.

23. The method of claim 14 wherein said composition is applied to a wound on the external body surface.
